(19) United States
(12) Patent Application Publication
(10) Pub. No.: US 2026/0200930 A1
(43) Pub. Date: Jul. 16, 2026

(54) PRODRUGS OF TEMOZOLOMIDE TO FACILITATE DELIVERY INTO BRAIN TUMORS

(71) Applicant: H. LEE MOFFITT CANCER CENTER AND RESEARCH INSTITUTE, INC., Tampa, FL (US)

(72) Inventors: Andrii MONASTYRSKYI, Lutz, FL (US); Michael VOGELBAUM, Tampa, FL (US); Derek DUCKETT, Land O' Lakes, FL (US)

(21) Appl. No.: 19/137,055

(22) PCT Filed: Dec. 8, 2023
(86) PCT No.: PCT/US2023/083059

§ 371 Date: Jun. 9, 2025

Related U.S. Application Data
(60) Provisional application No. 63/431,427, filed on Dec. 9, 2022.

(51) Int. Cl. C07D 487/04; A61K 31/496; A61K 31/5377

(52) U.S. Cl. CPC C07D 487/04; A61K 31/496; A61K 31/5377

(57) ABSTRACT

Disclosed are prodrugs of temozolomide (TMZ) of Formula I and Formula II that can have increased solubility and/or increased permeability across the blood brain barrier as compared to TMZ. Methods of making and using the disclosed prodrugs are also described.

### CROSS-REFERENCE TO RELATED APPLICATIONS

This application claims the benefit of priority to U.S. Provisional Application No. 63/431,427 filed Dec. 9, 2022, the disclosure of which is incorporated herein by reference in its entirety.

### TECHNICAL FIELD

This disclosure relates to compounds useful in treating medical disorders and, more particularly, to prodrugs of temozolomide that are useful in treating brain tumors.

### BACKGROUND

Primary malignant brain tumors, also known as gliomas, remain an incurable form of cancer despite decades of development of novel treatments and treatment strategies. One aspect of this disease, which also impacts other malignancies of the brain (e.g., metastatic cancer to the brain, lymphoma, non-glioma brain tumors, etc.), is the presence of a blood-brain-barrier (BBB) that prevents most therapeutic molecules from reaching tumor cells/tissues in the brain at effective concentrations. The few antineoplastic drugs that can penetrate the BBB produce intratumoral and intracerebral concentrations that are substantially lower than their plasma concentrations and, as a consequence, are only partially effective.

One such drug is temozolomide (TMZ), an oral chemotherapy, which has become the most commonly used anti-glioma therapy. This is despite its limited clinical activity, as its brain concentration is only about 40% of the highest plasma concentration that can safely be obtained. Multiple approaches have been taken to modify systemically administered (oral or intravenous) drugs to increase their BBB permeability, and most often, these modifications have resulted in a loss of therapeutic activity. Another approach to overcoming the BBB is to bypass it, delivering therapeutics directly into brain and brain tumor tissue. The approach of direct brain delivery requires that the infused drug be water soluble so that it can be easily formulated in aqueous solutions. Common methods to solubilize hydrophobic molecules involve excipients, none of which are safe for direct administration to brain tissue.

There is a clear need for further therapeutics capable of treating brain tumors, particularly those that resolve the issues involving the BBB. This disclosure addresses this, as well as other needs.

### SUMMARY

In accordance with the purposes of the disclosed materials and methods, as embodied and broadly described herein, the disclosed subject matter, in one aspect, relates to compounds, compositions, and methods of making and using compounds and compositions.

In some aspects, the disclosed subject matter relates to prodrugs of temozolomide (TMZ). The disclosed prodrugs can have increased solubility as compared to TMZ. IN other aspects, the disclosed prodrugs can have increased permeability across the blood-brain barrier as compared to TMZ.

In one aspect, a compound of Formula I or Formula II is provided as described herein, or a pharmaceutically acceptable salt or derivative thereof.

In another aspect, a pharmaceutical composition is provided comprising a compound described herein, or a pharmaceutically acceptable salt or derivative thereof, and a pharmaceutically acceptable carrier or excipient.

In another aspect, a method is provided for treating cancer in a subject in need thereof, comprising administering to the subject a therapeutically effective amount of a compound described herein or a pharmaceutically acceptable salt or derivative thereof.

In another aspect, a method is provided of killing a tumor cell in a subject in need thereof, the method comprising administering to the subject a therapeutically effective amount of a compound described herein, or a pharmaceutically acceptable salt or derivative thereof.

In another aspect, a method is provided for treating a tumor of the central nervous system in a subject in need thereof comprising administering to the subject a therapeutically effective amount of a compound described herein or a pharmaceutically acceptable salt or derivative thereof.

In another aspect, a method is provided for treating a brain tumor in a subject in need thereof comprising intracerebral administration to the subject of a therapeutically effective amount of a compound described herein or a pharmaceutically acceptable salt or derivative thereof.

Additional advantages will be set forth in part in the description that follows, and in part, they will be obvious from the description or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

The accompanying figures, which are incorporated in and constitute a part of this specification, illustrate several aspects described below.

FIG. 1 is a graph of growth inhibition (GI₅₀) in a U251 cell line for temozolomide (TMZ), SCC-024, SCC-025, SCC-033, SCC-026, and SCC-127.

FIG. 2 is a graph of prodrug stability for SCC-127, SCC-025, SCC-024, and SCC-033 as compared to TMZ at pH 7.4.

FIG. 3 is a graph of prodrug stability for SCC-127, SCC-025, SCC-024, and SCC-033 as compared to TMZ at pH 4.0.

FIG. 4 is a graph of prodrug stability for SCC-127, SCC-025, SCC-024, and SCC-033 as compared to TMZ at pH 2.0.

FIG. 5 is a graph of 5-aminoimidazole-4-carboxamide (AIC) appearance for SCC-127, SCC-025, SCC-024, and SCC-033 as compared to TMZ at pH 7.4.

FIG. 6 is a photograph depicting the thermodynamic solubility (50 mg/mL) of TMZ (left) and SCC-127 (right).

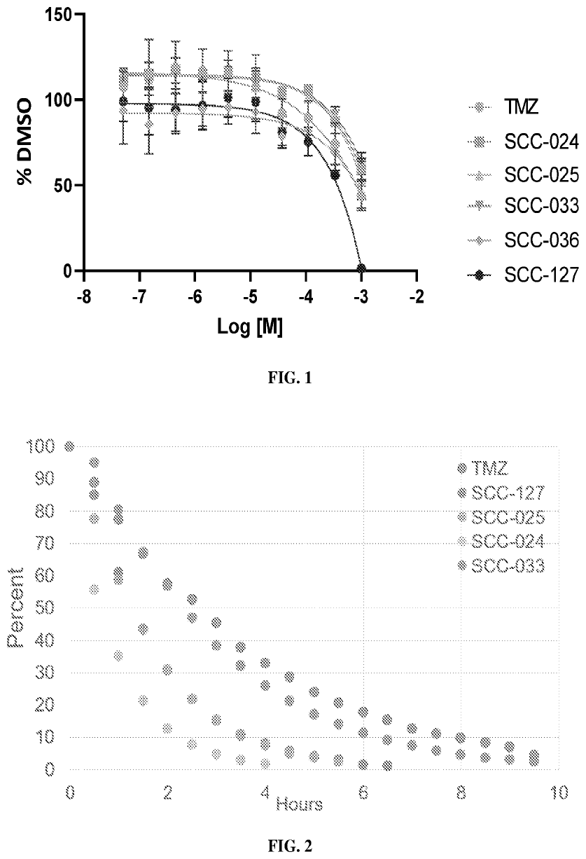

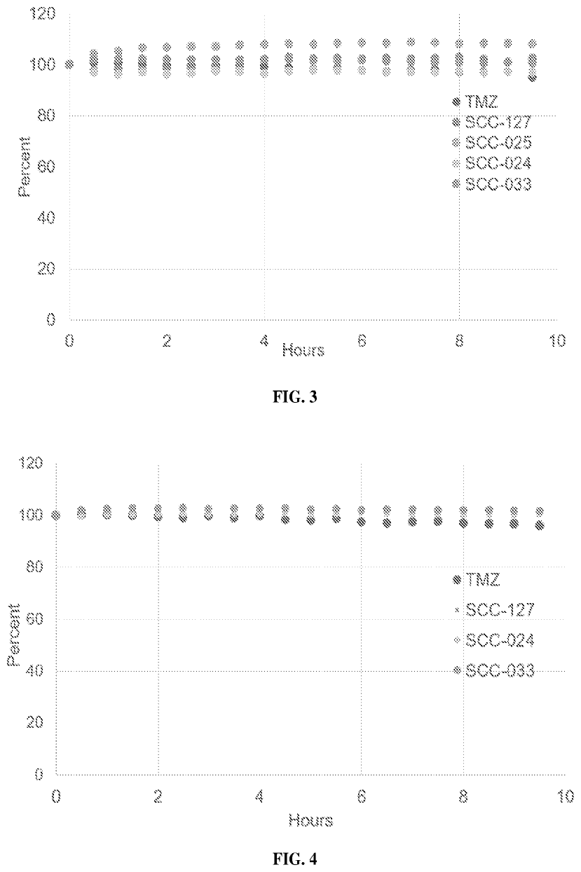

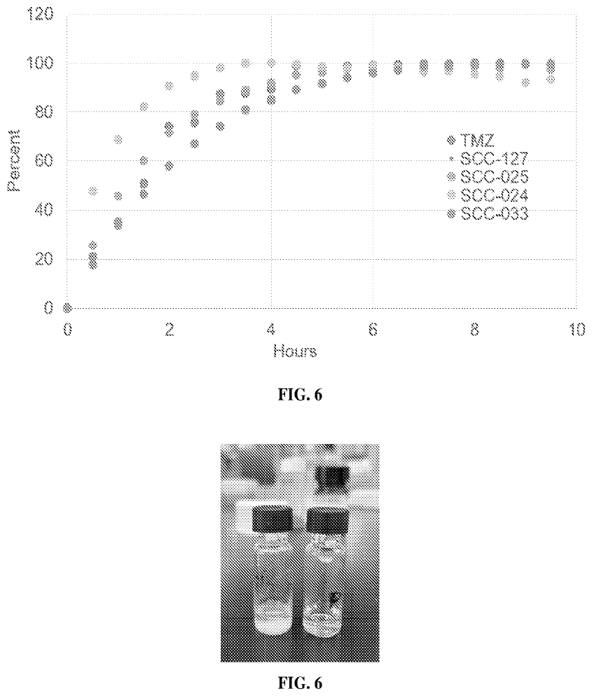

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present disclosure is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ ‘less than y,’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single-dose compositions can contain such amounts or submultiples thereof to make up the daily dose. The individual physician can adjust the dosage in the event of any contraindications. It is generally preferred that a maximum dose of the pharmacological agents of the disclosure (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition such as a tumor. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriately effective amount will be readily determined by one of ordinary skill in the art using only routine experimentation. Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-)configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-)form is equivalent, for compounds that undergo epimerization in vivo, to administration of the compound in its (S-)form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C=O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., =O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and/or can be formulated into a form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges, as used herein, indicate an alkyl group with the length of each member of the range described as an independent species. For example, C₁-C₆alkyl, as used herein, indicates an alkyl group having from 1, 2, 3, 4, 5, or 6 carbon atoms and is intended to mean that each of these is described as an independent species, and C₁-C₄alkyl, as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups, such as heteroatoms, as in —O—C₀-C₄alkyl(C₃-C₇cycloalkyl). Examples of alkyl include but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein.

“Cycloalkyl” is a saturated or partially unsaturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges, as used herein, indicate an alkynyl group, with each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group, as defined above, covalently bound through an oxygen bridge (—O—). Examples of alkoxy include but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group, as defined above, with the indicated number of carbon atoms covalently bound through a sulfur bridge (—S—).

“Alkanoyl” is an alkyl group, as defined above, covalently bound through a carbonyl (C═O) bridge. The carbonyl carbon is included in the number of carbons. For example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring that contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

### Compounds

In some aspects, the present disclosure provides compounds that are prodrugs of temozolomide (TMZ). In some aspects, the compounds described herein can have increased solubility and/or increased permeability across the blood-brain barrier as compared to TMZ.

In one aspect, a compound is provided of Formula II

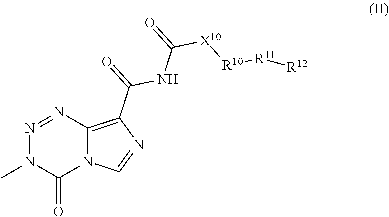

or a pharmaceutically acceptable salt or derivative thereof;
wherein:
X¹⁰ is a bond or selected from —NH— and —O—;
R¹⁰ is selected from C₁-C₆ alkyl optionally substituted with one or more groups selected from Z;
R¹¹ is a bond or —S—S—(C₁-C₆ alkyl) optionally substituted with one or more groups selected from Z;
R¹² is selected from —NR^{x}R^{y}, 3- to 8-membered monocyclic or bicyclic heterocycle, and 5- to 10-membered monocyclic or bicyclic heteroaryl, each of which may be optionally substituted with one or more groups selected from Z;
Z is selected from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R'S(O)—(C₀-C₃ alkyl)-, and R^{z}S(O)₂—(C₀-C₃ alkyl)-, each of which may be substituted by Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

In some aspects, X¹⁰ is a bond. In some aspects, X¹⁰ is —NH—. In some aspects, X¹⁰ is —O—.

In some aspects, R¹⁰ is selected from C₂-C₄ alkyl. In some aspects, R¹⁰ is C₂ alkyl.

In some aspects, R¹³ is a bond. In some aspects, R¹¹ is —S—S—(C₁-C₆ alkyl)-. In some aspects, R¹ is —S—S—(C₂ alkyl)-.

In some aspects, R¹² is —NH₂. In some aspects, R¹² is 3- to 8-membered monocyclic or bicyclic heterocycle. In some aspects, R¹² is 6-membered monocyclic heterocycle. In some aspects, R¹² is piperazinyl or morpholino optionally substituted with one or more groups selected from Z.

In another aspect, a compound is provided of Formula I

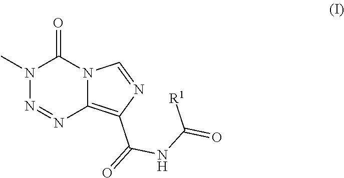

or a pharmaceutically acceptable salt or derivative thereof;
wherein R¹ is selected from C₁-C₈alkyl, C₂-C₅alkenyl, C₂-C₈alkynyl, C₃-C₈cycloalkyl, C₃-C₈cycloalkenyl, C₃-C₈heterocycle, OC₁-C₅alkyl, OC₂-C₈alkenyl, OC₂-C₈alkynyl, OC₃-C₅cycloalkyl, OC₃-C₅cycloalkenyl, OC₃-C₅heterocycle, NHC₁-C₅alkyl, NHC₂-C₅alkenyl, NHC₂-C₅alkynyl, NHC₃-C₅cycloalkyl, NHC₃-C₅cycloalkenyl, NHC₃-C₈heterocycle, SC₃-C₈alkyl, SC₂-C₈alkenyl, SC₂-C₈alkynyl, SC₃-C₈cycloalkyl, SC₃-C₈cycloalkenyl, or SC₃—SC₈heterocycle, each of which is optionally substituted with R²;
R² is OH, OR³, CO₂H, CO₂R³, OC(O)R³, SH, SR₃, S₂R³, SOR³, SO₂R³, NH₂, NH₃⁺, NHR³, NH₂R³⁺, C(O)NH₂, C(O)NH₃⁺, NHCO₂H, NHCO₂R³, SO₂NHR³, NHSO₂R³, unsubstituted C₃-C₅heterocycle, or C₃-C₅heterocycle substituted with R³; and
R³ is C₁-C₆alkyl optionally substituted with OH, NH₂, or NH₃⁺.

In specific aspects, the disclosed compounds can have R¹ substituted with R². In other aspects, R¹ is not substituted with R². In still another aspect, R¹ can be OC₁-C₈alkyl substituted with R². For example, R¹ can be a OC₂alkyl optionally substituted with R².

In other aspects, R² can be NH₂, NH₃⁺, NHR³, or NH₂R³⁺. In still other aspects, R² can be unsubstituted C₃-C₅heterocycle or C₃-C₅heterocycle substituted with R³.

In specific aspects, R² can be S₂R³, NH₂, NH₃⁺, NHR³, or NH₂R³⁺, unsubstituted C₃-C₈heterocycle, or C₃-C₅heterocycle substituted with R³, preferably when R¹ is OC₁-C₈alkyl.

In another aspect, a compound is provided selected from:

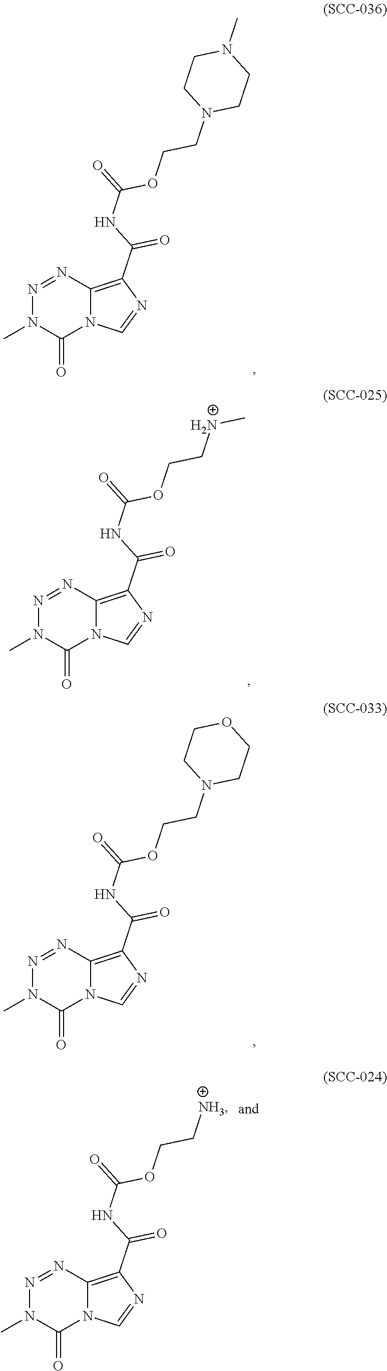

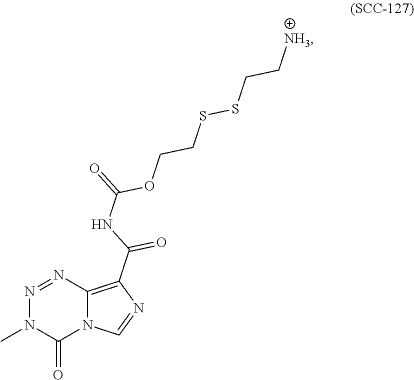

or a pharmaceutically acceptable salt or derivative thereof.

The compounds described herein are prodrugs of temozolomide (TMZ). TMZ is spontaneously hydrolyzed at physiological pH to 3-methyl-(triazen-1-yl)imidazole-4-carboxamide (MTIC), which further splits into monomethyl hydrazine, the likely active methylating agent which provides the observed therapeutic, and 4-aminoimidazole-4-carboxamide (AIC). As illustrated in FIG. 5, the prodrugs described herein hydrolyze at pH 7.4 to provide AIC in a similar fashion to TMZ.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like) or by reacting free base forms of these compounds with a stoichiometric amount of the appropriate acid. Such reactions are typically carried out in water, in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts that are acceptable for human consumption and the quaternary ammonium salts of the parent compound formed, for example, from inorganic or organic salts. Examples of such salts include but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Suitable counterions found in pharmaceutically acceptable salts described herein include but are not limited to, cations such as calcium, chloroprocaine, choline, diethanolamine, ethanolamine, ethylenediamine, meglumine, potassium, procaine, sodium, triethylamine, and zinc, and anions such as acetate, aspartate, benzenesulfonate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, and tosylate. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

The present disclosure also includes compounds described herein with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹³C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example, ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F-labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

By way of general example and without limitation, isotopes of hydrogen, for example, deuterium (²H) and tritium (³H), may optionally be used anywhere in described structures that achieve the desired result. Alternatively, or in addition, isotopes of carbon, e.g., ¹³C and ¹⁴C, may be used. In one aspect, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance and, in an aspect, is enough to alter a detectable property of the compounds as a drug in a human.

The compounds of the present disclosure may form a solvate with solvents (including water). Therefore, in one aspect, the disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates in accordance with the disclosure include those wherein the solvent of crystallization may be isotopically substituted, e.g., D₂O, d₆-acetone, or d₆-DMSO. A solvate can be in a liquid or solid form.

### Pharmaceutical Compositions

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art, including, for example, oral and parenteral routes of administration. As used herein, the term “parenteral” includes subcutaneous, intradermal, intravenous, intramuscular, intraperitoneal, and intrasternal administration, such as by injection. Administration of the active components of their compositions can be a single administration or at continuous and distinct intervals as can be readily determined by a person skilled in the art.

Compositions, as described herein, comprising an active compound and a pharmaceutically acceptable carrier or excipient of some sort, may be useful in a variety of medical and non-medical applications. For example, pharmaceutical compositions comprising an active compound and an excipient may be useful for the treatment or prevention of a brain tumor in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include but are not limited to, any non-toxic, inert solid, semisolid, or liquid filler, diluent, encapsulating material, or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, the time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art, such as for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed, including synthetic mono- or diglycerides. In addition, fatty acids such as oleic acid are used in the preparation of injectables. The injectable composition can be sterilized, for example, by filtration through a bacteria-retaining filter or by incorporating sterilizing agents in the form of sterile solid compositions, which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or by dispersing the particles in a polymer matrix or gel.

### Methods of Use

Further provided herein are methods of treating or preventing cancer in a subject, comprising administering to the subject an effective amount of a compound or composition as disclosed herein. The methods can further comprise administering a second compound or composition, such as for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can have an oncological disorder. Oncological disorders include, but are not limited to, cancer and/or tumors of the brain. Specific cancers contemplated for treatment include glioblastoma and refractory anaplastic astrocytoma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, primary or metastatic central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, metastatic melanoma, squamous cell carcinoma, small cell carcinoma, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenstrom's macroglobulinemia, and Wilms' tumor.

In some aspects, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during, or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

In one aspect, a method is provided for treating a tumor of the central nervous system (e.g., a brain tumor) in a subject in need thereof comprising administering to the subject a therapeutically effective amount of a compound described herein. In some aspects, administering to the subject comprises direct delivery of a therapeutically effective amount of a compound described herein to the central nervous system, i.e., via intracerebral, intracerebroventricular, or intrathecal administration. In some aspects, administering to the subject comprises direct delivery of a therapeutically effective amount of a compound described herein to the brain parenchyma, the spinal cord parenchyma, or the meninges.

In some particular aspects, a method is provided of treating a brain tumor in a subject in need thereof comprising intracerebral administration of a therapeutically effective amount of a compound described herein. In some aspects, intracerebral administration comprises direct delivery to the brain parenchyma.

In some aspects, the tumor of the central nervous system comprises a glioma, glioneuronal tumor, or a neuronal tumor.

In some aspects, the tumor comprises an adult-type diffuse glioma, such as: astrocytoma, IDH-mutant; oligodendroglioma, IDH-mutant and 1p/19q-codeleted; and glioblastoma.

In some aspects, the tumor comprises a pediatric-type diffuse low-grade glioma, such as: diffuse astrocytoma, MYB- or MYBL1-altered; angiocentric glioma; polymorphous low-grade neuroepithelial tumor of the young (PLNTY); and diffuse low-grade glioma, MAPK pathway-altered.

In some aspects, the tumor comprises a pediatric-type diffuse high-grade glioma, such as: diffuse midline glioma, H3 K27-altered; diffuse hemispheric glioma, H3 G34-mutant; diffuse pediatric high-grade glioma, H3-wildtype and IDH-wildtype; and infant-type hemispheric glioma.

In some aspects, the tumor comprises a circumscribed astrocytic glioma, such as: pilocytic astrocytoma; high-grade astrocytoma with piloid features; pleomorphic xanthoastrocytoma; suependymal giant cell astrocytoma; choroid glioma; and astroblastoma, MN-1 altered.

In some aspects, the tumor comprises a glioneuronal or neuronal tumor, such as: ganglioglioma; desmoplastic infantile ganglioglioma/desmoplastic infantile astrocytoma; dysembryoplastic neuroepithelial tumor; diffuse glioneuronal tumor with oligodendroglioma-like features and nuclear clusters; papillary glioneuronal tumor; rosette-forming glioneuronal tumor; myxoid glioneuronal tumor; diffuse leptomeningeal glioneuronal tumor; gangliocytoma; multinodular and vacuolating neuronal tumor; dysplastic cerebellar gangliocytoma (Lhermitte-Duclos disease); central neurocytoma; extraventricular neurocytoma; and cerebellar liponeurocytoma.

In some aspects, the tumor comprises an ependymal tumor, such as: supratentorial ependymoma (ZFTA fusion-positive or YAPI fusion-positive); posterior fossa ependymoma; posterior fossa ependymoma, group PFA; posterior fossa ependymoma, group PFB; spinal ependymoma; spinal ependymoma, MYCN-amplified; myxopapillary ependymoma; and subependymoma.

In some aspects, the tumor comprises a choroid plexus tumor, such as: choroid plexus papilloma; atypical choroid plexus papilloma; and choroid plexus carcinoma.

In some aspects, the tumor comprises an embryonal tumor, such as: medulloblastoma; atypical teratoid/rhabdoid tumor; cribiform neuroepithelial tumor; embryonal tumor with multilayered rosettes; CNS neuroblastoma, FOXR2-activated; and CNS tumor with BCOR internal tandem duplication.

In some aspects, the tumor comprises a pineal tumor, such as: pineocytoma; pineal parenchymal tumor of intermediate differentiation; pineoblastoma; papillary tumor of the pineal region; and desmoplastic myxoid tumor of the pineal region, SMARCB1-mutant.

In some aspects, the tumor comprises a cranial or paraspinal nerve tumor, such as: schwannoma; neurofibroma; perineurioma; hybrid nerve sheath tumor; malignant melanotic nerve sheath tumor; malignant peripheral nerve sheath tumor; and paraganglioma.

In some aspects, the tumor may comprise a meningioma, such as: meningioma; meningothelial meningioma; fibrous meningioma; transitional meningioma; psammomatous meningioma; angiomatus meningioma; microcystic meningioma; secretory meningioma; lymphoplasmacyte-rich meningioma; metaplastic meningioma; chordoid meningioma; clear cell meningioma; atypical meningioma; papillary meningioma; rhabdoid meningioma; and anaplastic (malignant) meningioma.

In some aspects, the tumor comprises a mesenchymal, non-meningothelial tumor, including: a soft tissue tumors, such as a fibroblastic and myofibroblastic tumor, including a solitary fibrous tumor; a vascular tumor such as a hemangioma or vascular malformation or a hemangioblastoma; a skeletal muscle tumor such as rhabdomyosarcoma; an uncertain differentiation, such as: an intracranial mesenchymal tumor, FET-CREB fusion-positive; CIC-rearranged sarcoma; a primary intracranial sarcoma, DICER1-mutant; or Ewing sarcoma; a chondro-osseous tumor, such as: a chondrogenic tumors, including mesenchymal chondrosarcoma or chondrosarcoma; or a notochordal tumor, such as chordoma (including poorly differentiated chordoma).

In some aspects, the tumor comprises a melanocytic tumor, such as a diffuse meningeal melanocytic neoplasm, including meningeal melanocytosis and meningeal melanomatosis, or a circumscribed meningeal melanocytic neoplasm, including meningeal melanocytoma or meningeal melanoma.

In some aspects, the tumor comprises a hematolymphoid tumor, including: a lymphoma, such as CNS lymphoma (e.g., primary diffuse large B-cell lymphoma of the CNS, immunodeficiency-associated CNS lymphoma, lymphomatoid granulomatosis, or intravascular large B-cell lymphoma) or a rare lymphoma in the CNS (e.g., MALT lymphoma of the dura, other low-grade B-cell lymphoma of the CNS, anaplastic large cell lymphoma (ALK+/ALK−), or a T-cell and NK/T-cell lymphoma) or a histiocytic tumor, such as Erdheim-Chester disease, Rosai-Dorfman disease, juvenile xanthogranuloma, Langerhans cell histiocytosis, or histiocytic sarcoma.

In some aspects, the tumor comprises a germ cell tumor, such as: mature teratoma; immature teratoma; teratoma with somatic-type malignancy; germinoma; embryonal carcinoma; yolk sac tumor; choriocarcinoma; or mixed germ cell tumor.

In some aspects, the tumor comprises a tumor of the sellar region, such as: adamantinomatous craniopharyngioma; papillary craniopharyngioma; pituicytoma, granular cell tumor of the sellar region, and spindle cell oncocytoma; pituitary adenoma/PitNET; or pituitary blastoma.

In some aspects, the tumor comprises metastases to the central nervous system, including metastases to the brain and spinal cord parenchyma or metastases to the meninges.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity and in vivo activity in animal models. Methods for the extrapolation of effective dosages in mice and other animals to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The dosage can be adjusted by the individual physician in the event of any counterindications. Dosage can vary and can be administered in one or more dose administrations daily, for one or several days.

The disclosed compounds can be administered either sequentially or simultaneously in separate or combined pharmaceutical formulations. When one or more of the disclosed compounds are used in combination with a second therapeutic agent, the dose of each compound can be either the same or differ from that when the compound is used alone. Appropriate doses will be readily appreciated by those skilled in the art.

The term “administration” and variants thereof (e.g., “administering” a compound) in reference to a compound disclosed herein means introducing the compound into the system of the patient in need of treatment. When a compound disclosed herein is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds and compositions containing them can be accomplished by any suitable method and technique presently or prospectively known to those skilled in the art. For example, the disclosed compounds can be formulated in a physiologically- or pharmaceutically-acceptable form and administered by any suitable route known in the art, including, for example, oral, nasal, rectal, topical, and parenteral routes of administration. As used herein, the term parenteral includes subcutaneous, intradermal, intravenous, intramuscular, intraperitoneal, intraparenchymal, intratumoral, and intrasternal administration, such as by injection. Administration of the disclosed compounds or compositions can be a single administration or at continuous or distinct intervals as can be readily determined by a person skilled in the art. In specific aspects, the compounds and compositions disclosed herein can be administered by intraparenchymal administration, where, e.g., the material is injected directly into the brain tissue. In other aspects, the compounds and compositions disclosed herein can be administered intratumorally, where the material is administered directly into a tumor.

The compounds disclosed herein and compositions comprising them can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspensions, suppositories, injectable and infusible solutions, and sprays. The preferred form depends on the intended mode of administration and therapeutic application. The compositions also preferably include conventional pharmaceutically-acceptable carriers and diluents, which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient, and aqueous and nonaqueous sterile suspensions, which can include suspending agents and thickening agents. The formulations can be presented in unit-dose or multi-dose containers, for example, sealed ampoules and vials, and can be stored in a freeze-dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art, having regard to the type of formulation in question.

Compounds disclosed herein and compositions comprising them can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth, optionally in combination with a pharmaceutically acceptable carrier such as an inert diluent. Compounds and compositions disclosed herein can be systemically administered, such as intravenously or orally, optionally in combination with a pharmaceutically acceptable carrier, such as an inert diluent or an assimilable edible carrier for oral delivery. They can be enclosed in hard or soft shell gelatin capsules, compressed into tablets, or incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch, or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain the active compound, sucrose or fructose as a sweetening agent, methyl and propylparabens as preservatives, a dye, and a flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, compounds and agents disclosed herein can be applied as a liquid or solid. However, it will generally be desirable to administer them topically to the skin as compositions, in combination with a dermatologically acceptable carrier, which can be a solid or a liquid. Compounds, agents, and compositions disclosed herein can be applied topically to a subject's skin to reduce the size (and can include complete removal) of malignant or benign growths or to treat an infection site. Compounds and agents disclosed herein can be applied directly to the growth or infection site. Preferably, the compounds and agents are applied to the growth or infection site in a formulation, such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina, and the like. Useful liquid carriers include water, alcohols, glycols, or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like for application directly to the skin of the user. Examples of useful dermatological compositions that can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity and in vivo activity in animal models. Methods for the extrapolation of effective dosages in mice and other animals to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical, or parenteral administration, comprising an amount of a compound, constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors, including the condition (health) of the subject, the body weight of the subject, kind of concurrent treatment, if any, frequency of treatment, therapeutic ratio, as well as the severity and stage of the pathological condition.

For the treatment of oncological disorders, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.) and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants, as described herein. In another aspect, a kit includes one or more anti-cancer agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

In view of the described compounds, compositions, and methods, hereinbelow are described certain more particular aspects of the disclosure. These particularly recited aspects should not, however, be interpreted to have any limiting effect on any different claims containing different or more general teachings described herein or that the “particular” aspects are somehow limited in some way other than the inherent meanings of the language and formulas literally used therein.

Aspect 1. A compound of Formula II

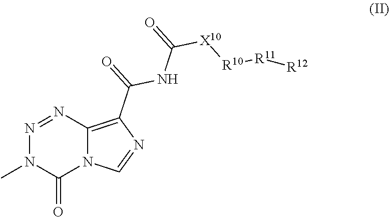

or a pharmaceutically acceptable salt or derivative thereof;
wherein:
X¹⁰ is a bond or selected from —NH— and —O—;
R¹⁰ is selected from C₁-C₆ alkyl optionally substituted with one or more groups selected from Z;
R¹¹ is a bond or —S—S—(C₁-C₆ alkyl) optionally substituted with one or more groups selected from Z;
R¹² is selected from —NR^{x}R^{y}, 3- to 8-membered monocyclic or bicyclic heterocycle, and 5- to 10-membered monocyclic or bicyclic heteroaryl, each of which may be optionally substituted with one or more groups selected from Z;
Z is selected from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R'S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted by Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

Aspect 2. The compound of claim 1, or a pharmaceutically acceptable salt or derivative thereof, wherein X¹⁰ is —O—.

Aspect 3. The compound of claim 1 or claim 2, or a pharmaceutically acceptable salt or derivative thereof, wherein R¹⁰ is C₂-C₄ alkyl.

Aspect 4. The compound of any one of claims 1-3, or a pharmaceutically acceptable salt or derivative thereof, wherein R¹⁰ is C₂ alkyl.

Aspect 5. The compound of any one of claims 1-4, or a pharmaceutically acceptable salt or derivative thereof, wherein R¹¹ is a bond.

Aspect 6. The compound of any one of claims 1-4, or a pharmaceutically acceptable salt or derivative thereof, wherein R¹¹ is —S—S—(C₁-C₆ alkyl)-.

Aspect 7. The compound of claim 6, or a pharmaceutically acceptable salt or derivative thereof, wherein R¹¹ is —S—S—(C₂ alkyl)-.

Aspect 8. The compound of any one of claims 1-7, or a pharmaceutically acceptable salt or derivative thereof, wherein R¹² is —NH₂.

Aspect 9. The compound of any one of claims 1-7, or a pharmaceutically acceptable salt or derivative thereof, wherein R¹² is 3- to 8-membered monocyclic or bicyclic heterocycle optionally substituted with one or more groups selected from Z.

Aspect 10. The compound of any one of claims 1-8, or a pharmaceutically acceptable salt or derivative thereof, wherein R¹² is 6-membered monocyclic heterocycle optionally substituted with one or more groups selected from Z.

Aspect 11. The compound of any one of claims 1-10, or a pharmaceutically acceptable salt or derivative thereof, wherein R¹² is piperazinyl or morpholino optionally substituted with one or more groups selected from Z.

Aspect 12. A compound having Formula I:

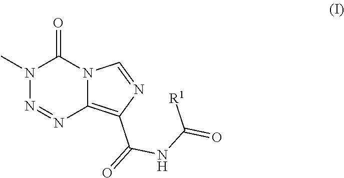

or a pharmaceutically acceptable salt or derivative thereof;
wherein:
R¹ is selected from C₁-C₅alkyl, C₂-C₅alkenyl, C₂-C₅alkynyl, C₃-C₅cycloalkyl, C₃-C₈cycloalkenyl, C₃-C₅heterocycle, OC₁-C₅alkyl, OC₂-C₅alkenyl, OC₂-C₅alkynyl, OC₃-C₈cycloalkyl, OC₃-C₈cycloalkenyl, OC₃-C₈heterocycle, NHC₁-C₅alkyl, NHC₂-C₈alkenyl, NHC₂-C₅alkynyl, NHC₃-C₅cycloalkyl, NHC₃-C₅cycloalkenyl, NHC₃-C₅heterocycle, SC₁-C₈alkyl, SC₂-C₅alkenyl, SC₂-C₅alkynyl, SC₃-C₈cycloalkyl, SC₃-C₅cycloalkenyl, or SC₃—SC₈heterocycle, each of which is optionally substituted with R²;
R² is OH, OR³, CO₂H, CO₂R³, OC(O)R³, SH, SR³, S₂R³, SOR³, SO₂R³, NH₂, NH₃⁺, NHR³, NH₂R³⁺, C(O)NH₂, C(O)NH₃⁺, NHCO₂H, NHCO₂R³, SO₂NHR³, NHSO₂R³, unsubstituted C₃-C₈heterocycle, or C₃-C₅heterocycle substituted with R³; and
R³ is C₁-C₅alkyl optionally substituted with OH, NH₂, or NH₃⁺.

Aspect 13. The compound of claim 12, or a pharmaceutically acceptable salt or derivative thereof, wherein R¹ is substituted with R².

Aspect 14. The compound of claim 12, or a pharmaceutically acceptable salt or derivative thereof, wherein R¹ is OC₁-C₈alkyl substituted with R².

Aspect 15. The compound of claim 13, or a pharmaceutically acceptable salt or derivative thereof, wherein R² is NH₂, NH₃⁺, NHR³, or NH₂R³⁺.

Aspect 16. The compound of claim 13, or a pharmaceutically acceptable salt or derivative thereof, wherein R² is unsubstituted C₃-C₅heterocycle or C₃-C₅heterocycle substituted with R³.

Aspect 17. The compound of claim 14, or a pharmaceutically acceptable salt or derivative thereof, wherein R² is S₂R³, NH₂, NH₃⁺, NHR³, or NH₂R³⁺, unsubstituted C₃-C₈heterocycle, or C₃-C₅heterocycle substituted with R³.

Aspect 18. The compound of claim 14, or a pharmaceutically acceptable salt or derivative thereof, wherein R³ is C₁-C₆alkyl.

Aspect 19. A compound selected from:

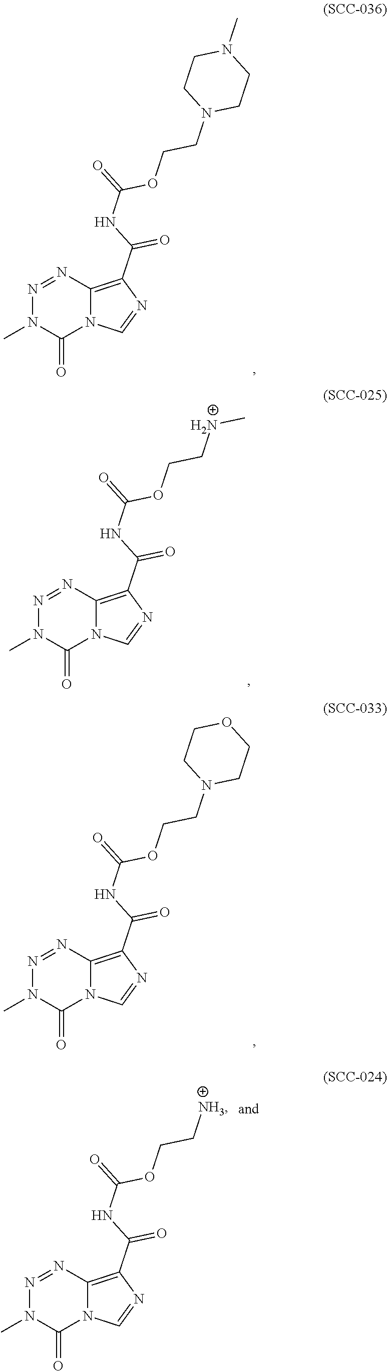

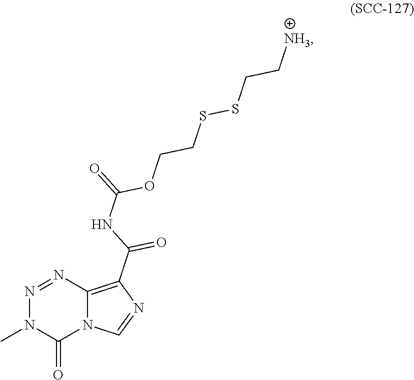

or a pharmaceutically acceptable salt or derivative thereof.

Aspect 20. A pharmaceutical composition comprising a compound of any one of claims 1-19, or a pharmaceutically acceptable salt or derivative thereof, and a pharmaceutically acceptable carrier or excipient.

Aspect 21. A method of treating cancer in a subject in need thereof, comprising administering to the subject a therapeutically effective amount of a compound of any one of claims 1-19, or a pharmaceutically acceptable salt or derivative thereof, or a pharmaceutical composition of claim 20.

Aspect 22. The method of claim 21, wherein the cancer is a tumor of the central nervous system.

Aspect 23. The method of claim 21 or claim 22, wherein the cancer is a glioma.

Aspect 24. A method of killing a tumor cell in a subject in need thereof, the method comprising administering to the subject a therapeutically effective amount of a compound of any one of claims 1-19, or a pharmaceutically acceptable salt or derivative thereof, or a pharmaceutical composition of claim 20.

Aspect 25. The method of claim 24, wherein the tumor cell is a central nervous system tumor cell.

Aspect 26. The method of claim 24 or claim 25, wherein the tumor cell is a glioma cell.

Aspect 27. A method for treating a tumor of the central nervous system in a subject in need thereof comprising administering to the subject a therapeutically effective amount of a compound of any one of claims 1-19, or a pharmaceutically acceptable salt or derivative thereof, or a pharmaceutical composition of claim 20.

Aspect 28. The method of claim 27, wherein the tumor of the central nervous system is a brain tumor.

Aspect 29. The method of claim 27 or claim 28, wherein administering to the subject comprises direct delivery to the central nervous system.

Aspect 30. The method of claim 29, wherein direct delivery to the central nervous system comprises intracerebral, intracerebroventricular, or intrathecal administration.

Aspect 31. The method of claim 27 or claim 28, wherein administering to the subject comprises direct delivery to the brain parenchyma, the spinal cord parenchyma, or the meninges.

Aspect 32. A method for treating a brain tumor in a subject in need thereof comprising intracerebral administration to the subject of a therapeutically effective amount of a compound of any one of claims 1-19, or a pharmaceutically acceptable salt or derivative thereof, or a pharmaceutical composition of claim 20.

Aspect 33. The method of claim 32, wherein intracerebral administration comprises direct delivery to the brain parenchyma.

Aspect 34. The method of any one of claims 21-33, further comprising exposing the patient to a therapeutically effective dose of ionizing radiation.

Aspect 35. The method of any one of claims 21-34, wherein the subject is a human.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### Examples

The following examples are set forth below to illustrate the compounds, compositions, and methods claimed herein, along with associated methods and results according to the disclosed subject matter. These examples are not intended to be inclusive of all aspects of the subject matter disclosed herein but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions, that can be used to optimize the product purity and yield obtained from the described process. Only reasonable and routine experimentation will be required to optimize such process conditions.

One example of the use of the disclosed subject matter is for its use to treat residual glioblastoma tumor cells that invade the brain surrounding a tumor mass. These cells cannot be removed safely, as they are surrounded, at a microscopic level, by functioning brain cells, and the surgical removal of the tissue containing both tumor and normal cells will lead to irreversible loss of neurological functioning. Direct delivery of the current form of temozolomide (TMZ) is not possible, as it is insoluble in water, and the concentration in brain tissue that is achieved after oral administration of TMZ is insufficient to prevent further growth and invasion of the residual glioblastoma tumor cells. A higher concentration of TMZ in the brain can be achieved only by direct delivery, and the present disclosure makes it possible to produce a sufficiently high dose of active TMZ in brain tissue that is lethal to the tumor cells.

### Synthesis of TMZ Prodrugs

General. All reagents were purchased from commercial suppliers and were used without further purification. Dichloromethane, diethyl ether, N,N-dimethylformamide and tetrahydrofuran were dried by being passed through a column of desiccant (activated A-1 alumina). Triethylamine and diisopropyl amine were purified by distillation from calcium hydride. Reactions were either monitored by thin layer chromatography or analytical LC-MS. Thin layer chromatography was performed on Kieselgel 60 F254 glass plates pre-coated with a 0.25 mm thickness of silica gel. TLC plates were visualized with UV light and/or by staining with ninhydrin solution. Normal phase column chromatography was performed on a Biotage Selekt automated flash system. Compounds were loaded onto pre-filled cartridges filled with KP-Sil 50 μm irregular silica. For microwave reactions, a Biotage Initiator Microwave system was used. Some of the final products were isolated by reverse-phase HPLC using Waters HPLC system with UV detector, with Atlantis T3 OBD Prep Column, 100Å, 5 μm, 19 mm×150 mm. Compounds were eluted using a gradient elution of 90/10 to 0/100 A/B over 20 min at a flow rate of 20.0 mL/min, where solvent A was water (+0.1% ammonium acetate) and solvent B was acetonitrile.

The structures of all compounds were verified via ¹H NMR, ¹³C NMR, ¹⁹F NMR and LCMS. The purity of isolated products was determined using an LC-MS instrument (Agilent 1290 Infinity series LC with single quadrupole MSD system, AP-ESI Ion Source) equipped with Kinetex® 1.7 μm C18 100 Å, LC Column 50×2.1 mm, Ea (Phenomenex) column. Elution was performed using the following conditions: 2% (v/v) acetonitrile (+0.1% FA) in 98% (v/v) H₂O (+0.1% FA), ramped to 98% acetonitrile over 4.0 min, and holding at 98% acetonitrile for 0.5 min with a flow rate of 0.6 mL/min; UV absorption was detected from 200 to 950 nm using a diode array detector. The purity of each compound was ≥95% based on this analysis.

NMR spectra were recorded at ambient temperature on a 500 MHz Bruker NMR spectrometer in DMSO-d6. All ¹H NMR data are reported in parts per million (ppm) downfield of TMS and were measured relative to the signals for dimethyl sulfoxide (2.50 ppm). All ¹³C NMR spectra are reported in ppm relative to the signals for dimethyl sulfoxide (39.5 ppm) with ¹H decoupled observation. ¹⁹F NMR experiments were performed with ¹H decoupling. Data for ¹H NMR are reported as follows: chemical shift (6, ppm), multiplicity (s=singlet, d=doublet, t=triplet, q=quartet, m=multiplet), integration, and coupling constant (Hz), whereas ¹³C NMR analyses were obtained at 125 Mhz and reported in terms of chemical shift. NMR data was analyzed and processed by using MestReNova software.

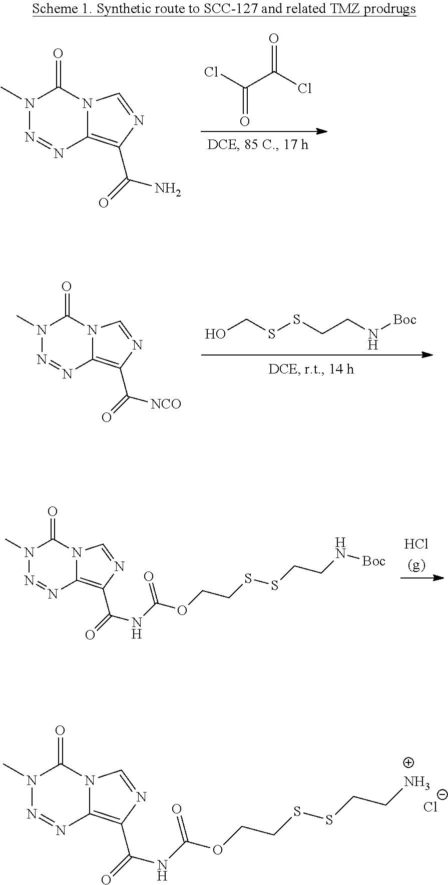

2-((3-(((3-methyl-4-oxo-3,4-dihydroimidazo[5,1-d][1,2,3,5]tetrazine-8-carbonyl)carbamoyl)oxy)propyl)thio)ethan-1-aminium chloride (SCC-127). To a 20 mL vial was added 25 mg of Boc-protected amine followed by 1.00 mL of ACN. 4.04 mL of concentrated HCL was added drop-wise to a 3-neck flask containing calcium chloride. This allowed the production of hydrogen chloride gas, which resulted in the de-protection of BOC. LCMS was done to monitor the progress of the reaction, and it was observed that complete de-protection occurred at 20 mins. This was followed by concentration under reduced pressure and washing with ether using petroleum ether. The white solid product was then allowed to dry under high vacuum to yield 20 mg (91% yield) of product. LCMS (m/z): 374 (M+1), Rₜ: 1.45 min. ¹H NMR (500 MHz, CDCl₃) δ 5.54-5.35 (m, 1H), 2.84-2.76 (m, 3H), 2.04-1.96 (m, 3H), 1.92 (ddd, J=8.5, 6.6, 1.8 Hz, 2H), 1.84 (ddd, J=8.3, 6.8, 1.7 Hz, 2H), 1.64 (t, J=7.4 Hz, 2H).

| Compound | Aq solubility [μM] |
|---|---|
| SCC-024 | >100 |
| SCC-025 | >100 |
| SCC-127 | >100 |

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps are also intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula II 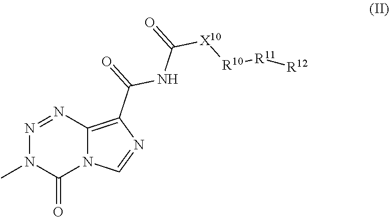
or a pharmaceutically acceptable salt or derivative thereof;
wherein:
X¹⁰ is a bond or selected from —NH— and —O—;
R¹⁰ is selected from C₁-C₆ alkyl optionally substituted with one or more groups selected from Z;
R¹¹ is a bond or —S—S—(C₁-C₆ alkyl) optionally substituted with one or more groups selected from Z;
R¹² is selected from —NR^{x}R^{y}, 3- to 8-membered monocyclic or bicyclic heterocycle, and 5- to 10-membered monocyclic or bicyclic heteroaryl, each of which may be optionally substituted with one or more groups selected from Z;
Z is selected from hydrogen, halo, nitro, cyano, azido, oxo, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀—C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted by Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, and thiol.

2-11. (canceled)

12. A compound having Formula I: 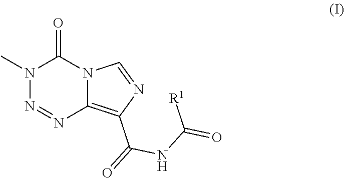
or a pharmaceutically acceptable salt or derivative thereof;
wherein:
R¹ is selected from C₁-C₈alkyl, C₂-C₈alkenyl, C₂-C₈alkynyl, C₃-C₈cycloalkyl, C₃-C₈cycloalkenyl, C₃-C₈heterocycle, OC₁-C₅alkyl, OC₂-C₈alkenyl, OC₂-C₈alkynyl, OC₃-C₈cycloalkyl, OC₃-C₈cycloalkenyl, OC₃-C₈heterocycle, NHC₁-C₈alkyl, NHC₂-C₈alkenyl, NHC₂-C₈alkynyl, NHC₃-C₈cycloalkyl, NHC₃-C₈cycloalkenyl, NHC₃-C₈heterocycle, SC₁-C₈alkyl, SC₂-C₈alkenyl, SC₂-C₈alkynyl, SC₃-C₈cycloalkyl, SC₃-C₈cycloalkenyl, or SC₃—SC₈heterocycle, each of which is optionally substituted with R²;
R² is OH, OR³, CO₂H, CO₂R³, OC(O)R³, SH, SR³, S₂R³, SOR³, SO₂R³, NH₂, NH₃⁺, NHR³, NH₂R³⁺, C(O)NH₂, C(O)NH₃⁺, NHCO₂H, NHCO₂R³, SO₂NHR³, NHSO₂R³, unsubstituted C₃-C₈heterocycle, or C₃-C₈heterocycle substituted with R³; and
R³ is C₁-C₆alkyl optionally substituted with OH, NH₂, or NH₃⁺.

13-18. (canceled)

19. A compound selected from: 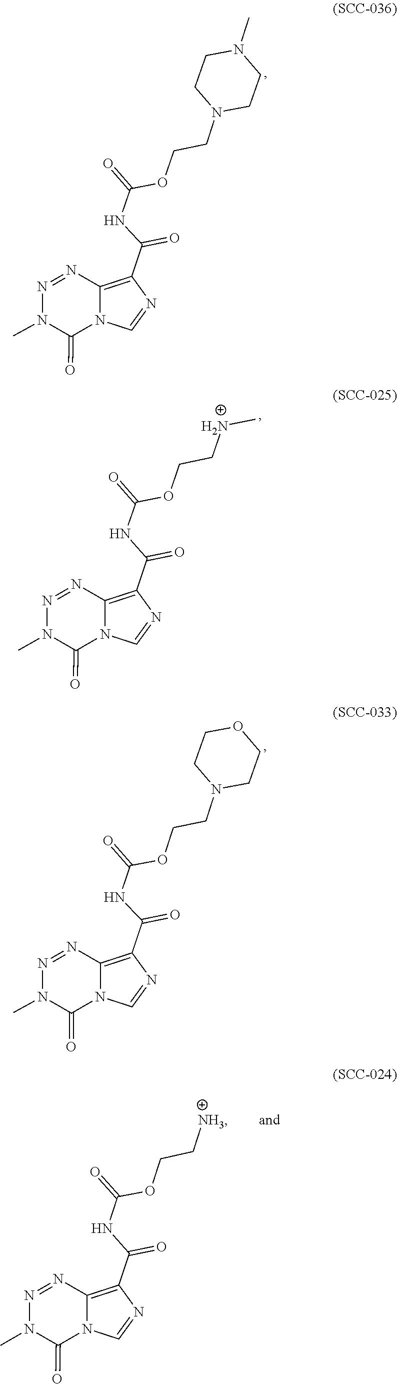 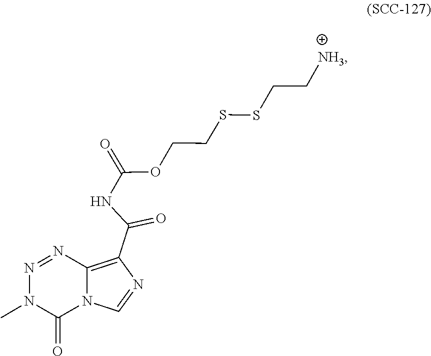
or a pharmaceutically acceptable salt or derivative thereof.

20. A pharmaceutical composition comprising a compound of claim 1, or a pharmaceutically acceptable salt or derivative thereof, and a pharmaceutically acceptable carrier or excipient.

21. A method of treating cancer in a subject in need thereof, comprising administering to the subject a therapeutically effective amount of a compound of claim 1, or a pharmaceutically acceptable salt or derivative thereof.

22-23. (canceled)

24. A method of killing a tumor cell in a subject in need thereof, the method comprising administering to the subject a therapeutically effective amount of a compound of claim 1, or a pharmaceutically acceptable salt or derivative thereof.

25-26. (canceled)

27. A method for treating a tumor of the central nervous system in a subject in need thereof comprising administering to the subject a therapeutically effective amount of a compound of claim 1, or a pharmaceutically acceptable salt or derivative thereof.

28-31. (canceled)

32. A method for treating a brain tumor in a subject in need thereof comprising intracerebral administration to the subject of a therapeutically effective amount of a compound of claim 1, or a pharmaceutically acceptable salt or derivative thereof, or a pharmaceutical composition of claim 20.

33-35. (canceled)

36. A pharmaceutical composition comprising a compound of claim 12, or a pharmaceutically acceptable salt or derivative thereof, and a pharmaceutically acceptable carrier or excipient.

37. A method of treating cancer in a subject in need thereof, comprising administering to the subject a therapeutically effective amount of a compound of claim 12, or a pharmaceutically acceptable salt or derivative thereof.

38. A method of killing a tumor cell in a subject in need thereof, the method comprising administering to the subject a therapeutically effective amount of a compound of claim 12, or a pharmaceutically acceptable salt or derivative thereof.

39. A method for treating a tumor of the central nervous system in a subject in need thereof comprising administering to the subject a therapeutically effective amount of a compound of claim 12, or a pharmaceutically acceptable salt or derivative thereof.

40. A method for treating a brain tumor in a subject in need thereof comprising intracerebral administration to the subject of a therapeutically effective amount of a compound of claim 12, or a pharmaceutically acceptable salt or derivative thereof.

41. A pharmaceutical composition comprising a compound of claim 19, or a pharmaceutically acceptable salt or derivative thereof, and a pharmaceutically acceptable carrier or excipient.

42. A method of treating cancer in a subject in need thereof, comprising administering to the subject a therapeutically effective amount of a compound of claim 19, or a pharmaceutically acceptable salt or derivative thereof.

43. A method of killing a tumor cell in a subject in need thereof, the method comprising administering to the subject a therapeutically effective amount of a compound of claim 19, or a pharmaceutically acceptable salt or derivative thereof.

44. A method for treating a tumor of the central nervous system in a subject in need thereof comprising administering to the subject a therapeutically effective amount of a compound of claim 19, or a pharmaceutically acceptable salt or derivative thereof.

45. A method for treating a brain tumor in a subject in need thereof comprising intracerebral administration to the subject of a therapeutically effective amount of a compound of claim 19, or a pharmaceutically acceptable salt or derivative thereof.
